(19) United States
(12) Patent Application Publication
(10) Pub. No.: US 2021/0145867 A1
(43) Pub. Date: May 20, 2021

(54) PLASMONIC ENHANCED MAGNETIC NANOPARTICLES HYPERTHERMIA

(71) Applicant: Board of Regents, The University of Texas System, Austin, TX (US)

(72) Inventors: Ahmed El-Gendy, El Paso, TX (US); Chunqiang Li, El Paso, TX (US)

(21) Appl. No.: 17/070,311

(22) Filed: Oct. 14, 2020

Related U.S. Application Data
(60) Provisional application No. 62/935,950, filed on Nov. 15, 2019.

(51) Int. Cl. A61K 33/26; A61K 33/38; A61K 33/242; A61K 9/51

(52) U.S. Cl. CPC A61K 33/26; A61K 33/38; B82Y 5/00; A61K 9/5192; A61K 33/242

(57) ABSTRACT

A method of plasmonic enhanced magnetic nanoparticles hyperthermia (PE-MNH) of M@X core/shell nanoparticles using laser energy. Up on laser exposure of the nanoparticles in solution, the plasmonic shell will heat up and isolate each particle in their own hydrodynamic shell that lead to reducing the inter-particle interaction of the magnetic nanoparticles. This will lead to disaggregated nanoparticle with high dispersity, free movement and rotation in solution as well as giant increase in SAR when the alternating magnetic field within clinical safety limits is applied. Application of this approach has the potential to revolutionize the current treatment regimens by replacing them with plasmonic enhanced magnetic nanoparticles hyperthermia therapy that is more effective, less toxic, and impact survival.

### CROSS REFERENCE TO RELATED APPLICATIONS

This application is related to and claims the benefit of priority of provisional U.S. Patent Application Ser. No. 62/935,950 filed Nov. 15, 2019, which is hereby incorporated by reference.

### BACKGROUND

Magnetic nanoparticles hyperthermia is a promising method for cancer therapy. Briefly, by injecting magnetic nanoparticles to tumors and then applying alternating magnetic field, localized heat will be induced from the particles to cause apoptotic death for the tumors in temperatures ranges of 42-46° C. Two mechanisms are in charge of such induced heat from the magnetic nanoparticles. One is due to particle motion in biological medium (Brown Relaxation) under applied AC (alternating current) field, and the second due to rotation of the magnetic moment of each particle (Neel Relaxation) under applied magnetic field.

Although magnetic hyperthermia research in the last few decades reveals promising results as alternative cancer therapy, it was soon realized that several operational constraints might limit the procedure's applicability. Large nanoparticles might not be able to escape the vasculature, the toxicity of some nanoparticles may be too high for use in humans, the magnitude of the magnetic field to which a human can be exposed is limited, and high-frequency magnetic fields can cause excessive unwanted direct tissue heating [1-2]. It has been suggested that for use in humans the product H·f, where H is the applied field magnitude and f is its frequency, should not be more than 5.8×10⁹ Am⁻¹ Hz [3]. While some of these limitations (e.g., particle size and toxicity) do not appear to be insurmountable obstacles, the inability of conventional magnetic nanoparticle systems to produce enough heat within the above-mentioned restrictions on the magnetic field's amplitude and frequency has been a problem for application of magnetic hyperthermia in clinical practice.

### SUMMARY

Clinical level new cancer therapy is presented using core/shell M@X magnetic nanoparticles with plasmonic enhanced magnetic hyperthermia, where M can be, e.g., Fe, Fe3O4, α-Fe2O3, γ-Fe2O3, Co, CoFe2O4, Ni, NiFe2O4, FeCo, FeNi, CoNi, or other magnetic elements or compounds and X can be, e.g., silver (Ag), gold (Au), or other suitable biocompatible plasmonic elements or compounds. In representative examples, Fe@Ag core/shells are synthesized using a room temperature wet chemistry method. The method is optimized to produce different sizes of Fe@Ag nanoparticles. The formation of Fe@Ag core/shell, small and mono-homogeneous size distribution of 8.3±1.4, and 13.8±1.4 nm is confirmed by means of XRD (x-ray diffraction), TEM (transmission electron microscopy), and SEM (scanning electron microscopy). The magnetic measurements reveal superparamagnetic behavior with high saturation magnetization of 145 and 141 emu/g for 8.3±1.4, and 13.8±1.4 nm respectively. The feasibility for hyperthermia is confirmed by measuring the dissipated heating power or specific absorption rate (SAR) of the samples solution under applied magnetic field and frequency. The hyperthermia experiments reveal representative maximum heating power of 227 W/g at 500 Oe and 164 kHz.

Femtosecond laser exposure to the sample's solution is used to enhance the particles dispersion in solution leading to more efficient localized heating power of the particles in solution. The SAR is measured after laser exposure and the value is increased to 1266 W/g at the same condition of field and frequency of 500 Oe and 164 kHz. This was observed at different field and frequencies with factor of 5-10 increase in SAR values. The data is confirmed by measuring the SAXS (small-angle X-ray scattering) for samples solution before and after laser exposure. SAXS data reveals that the particles after laser exposure shows more dispersion in solution than before laser exposure. This confirms the role of plasmonic surface of the silver shell under applied femtosecond laser to separate the hydrodynamic shell decreasing the interparticle interaction leading to free movement of isolated particles under applied field and frequency.

The biocompatibility of the selected optimized size of 8.3±1.4 nm is tested for in vitro leukemia and breast tumor cells for different particle's concentrations of 12.5-100 μg/ml. Cytotoxicity data confirm the biocompatibility of the 12.5 and 25 μg/ml doses. Herein, the feasibility of in vitro hyperthermia is performed for the optimized doses under applied therapeutic field and frequency of 400 Oe and 304 kHz followed by cell viability measurements. Data yielded no effect at all for the applied field and frequency on the solvent control and untreated cells. Moreover, there is no effect observed on particle-treated cells in the same day of hyperthermia experiment. However, the particle solutions with cells show apoptosis death for the leukemia cells after 3 and 6 days of in vitro hyperthermia experiment. The findings open new route of independent alternative cancer therapy using magnetic hyperthermia of core-shell Fe@Ag superparamagnetic nanoparticles with plasmonic enhancement.

The features and functions can be achieved independently in various embodiments of the present disclosure or may be combined in yet other embodiments in which further details can be seen with reference to the following description and drawings.

### BRIEF DESCRIPTION OF THE DRAWINGS

The novel features believed characteristic of the illustrative embodiments are set forth in the appended claims. The illustrative embodiments, however, as well as a preferred mode of use, further objectives and features thereof, will best be understood by reference to the following detailed description of an illustrative embodiment of the present disclosure when read in conjunction with the accompanying drawings, wherein:

FIG. 1 is a schematic illustration for exciting plasmon resonances of nanoparticles by applying ultrafast femtosecond laser pulses to enhance specific absorption rates of the particles in a biological solution;

FIG. 2(a-d) show TEM images and particles size distribution of the Fe@Ag nanoparticles;

FIG. 2(e) shows XRD pattern of the Fe@Ag nanoparticles with different sizes;

FIG. 2(f) shows the magnetization dependent of the field for different particles sizes revealing superparamagnetic behavior of Fe@Ag nanoparticles;

FIG. 3(a-b) show SAR dependence on external magnetic field and frequency (a) 8.3 nm and (b) 13.8 nm Fe@Ag nanoparticles dispersed in water;

FIG. 4 shows SAR dependence on external magnetic field and frequency for 8.3 nm Fe@Ag nanoparticles dispersed in water;

FIG. 5 shows SAR (scatter) and relaxation time (column/bar) dependence on external magnetic field at 144 kHz before and after 1 min laser exposure for 8.3 nm Fe@Ag nanoparticles;

FIG. 6(a-c) show SAXS experimental results.

FIG. 6a shows I(Q) curves for sample before (BF, orange symbols) and after (AF, dark grey) functionalization. The corresponding two-level UEP model for each sample (BF: blue, and AF: red) is plotted as well;

FIG. 6b is a Sketch showing the size of nanoparticles (D2) and aggregates (D1), and their corresponding mass fractal exponents, P2 and P1, respectively.

FIG. 6c is a sketch showing how the dimensions D1 and D2 change with functionalization, as well as the transition to independent particles which only exhibit surface fractal scattering (Pi>3).

FIG. 7a is a quantification of Cellular cytotoxicity (y-axis) using the fluorescent dye exclusion propidium iodide (PI) and flow cytometric assays;

FIG. 7(b-d) are Dot plots of cells treated for 6 days with a concentration gradient of MS011: B, 25 μg/ml; C, 50 μg/ml; and D, 100 μg/ml.

FIG. 7e is a dot plot of Cells treated with 1% v/v of PBS were used as a solvent control;

FIG. 7f is a dot plot of Untreated cells included as a negative control for cytotoxicity;

FIG. 7g is a dot plot of cells exposed for 24 h to 1 mM of H2O2 were incorporated as a positive control for cytotoxicity;

FIG. 8a is a quantification of Cellular cytotoxicity (y-axis) of Cells incubated after hyperthermia at field and frequency of 400 Oe and 304 kHz respectively; Cells treated with 1% v/v of PBS were used as a solvent control (d, h, l). Untreated cells were included as a negative control for cytotoxicity (e, i, m).

FIG. 8(b-e) are Dot plots of cells incubated for 0 days after hyperthermia experiment;

FIG. 8(f-i) are Dot plots of cells incubated for 0 days after hyperthermia experiment;

FIG. 8(j-m) are Dot plots of cells incubated for 0 days after hyperthermia experiment; and

FIG. 9(a-b) is a quantification of Cellular cytotoxicity of 8 nm Fe@Ag and 20 nm Fe@Au magnetic nanoparticles on MDA-MB231 breast cancer cell line.

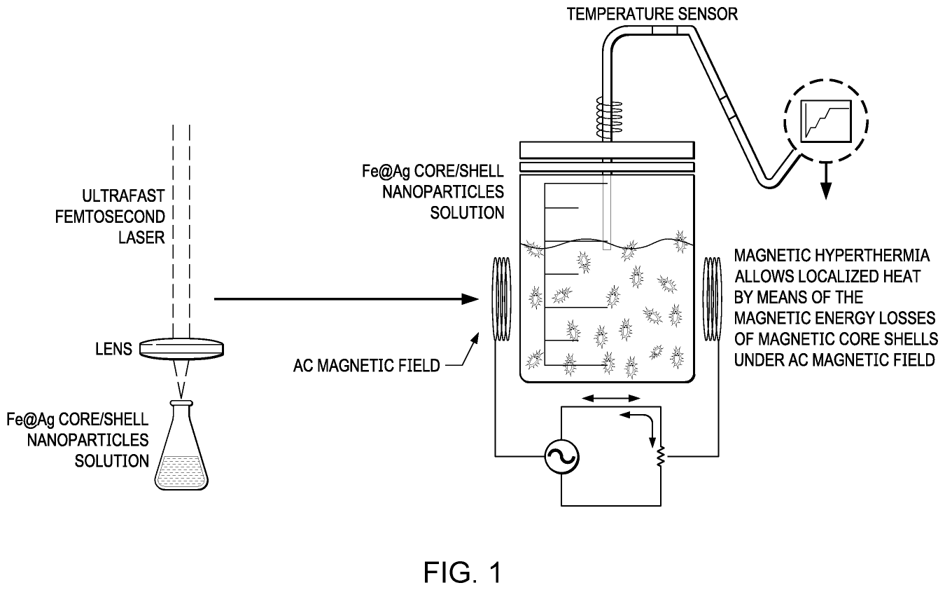

### DETAILED DESCRIPTION

The illustrative embodiments recognize and take into account a number of different considerations. For example, the illustrative embodiments recognize and take into account that the inability of conventional magnetic nanoparticle systems to produce enough heat within the above-mentioned restrictions on the magnetic field's amplitude and frequency has been a problem for application of magnetic hyperthermia in clinical practice.

As used herein, the term “nanoparticle” generally refers to matter in particulate form is a size range between 1 nanometers (nm) and 100 nm. As used herein, the term “particulate,” or contextual variants thereof, generally means being relating to or being in the form of separate particles. As used herein, the term “particle,” or contextual variants thereof, generally refers to a portion or fragment of matter. In some illustrative examples, particles can range in size from 5 μm to 300 μm, and can have any type of shape—for example, at least one of spherical, oblate, prolate, spheroid, cylindrical, orthorhombic, regular, irregular, or the like. Additionally, a quantity of particles comprising a same material can be provided in any number of sizes, or any number of shapes.

Efficient heat cannot be obtained because of two considerable technical challenge: formation of clusters and agglomerates of magnetic nanoparticles due to their inter-particle interaction which affects negatively the heating power of the magnetic nanoparticles; and low magnetic properties of the used magnetic nanoparticles which lead to weak response (slow rotation of magnetic moment) to the applied magnetic field as well as weak heating power. For efficient hyperthermia treatment, the magnetic nanoparticles to be imbedded into the cancer cell should have small monodispersed size, higher magnetization, higher heating power dissipated (e.g., SAR) and proper surface for the medical direction.

Clinical level new cancer therapy is presented using core/shell M@X magnetic nanoparticles with plasmonic enhanced magnetic hyperthermia, where M can be, e.g., Fe, Fe3O4, α-Fe2O3, γ-Fe2O3, Co, CoFe2O4, Ni, NiFe2O4, FeCo, FeNi, CoNi, or other magnetic elements or compounds and X can be, e.g., silver (Ag), gold (Au), or other suitable biocompatible plasmonic elements or compounds. In representative examples, Fe@Ag core/shells are synthesized using a room temperature wet chemistry method. The method is optimized to produce different sizes of Fe@Ag nanoparticles. The formation of Fe@Ag core/shell, small and mono-homogeneous size distribution of 8.3±1.4, and 13.8±1.4 nm is confirmed by means of XRD (x-ray diffraction), TEM (transmission electron microscopy), and SEM (scanning electron microscopy). The magnetic measurements reveal superparamagnetic behavior with high saturation magnetization of 145 and 141 emu/g for 8.3±1.4, and 13.8±1.4 nm respectively. The feasibility for hyperthermia is confirmed by measuring the dissipated heating power or specific absorption rate (SAR) of the samples solution under applied magnetic field and frequency. The hyperthermia experiments reveal representative maximum heating power of 227 W/g at 500 Oe and 164 kHz.

In one illustrative example, core/shell superparamagnetic (SPM) Fe@Ag nanoparticles are selected due to their monodispersity, biocompatibility, chemical stability, and high magnetic properties. Fe as magnetic core has high magnetic properties which allows for quick magnetic response (e.g., fast rotation of magnetic moment) to the AC magnetic field. Avoiding further oxidation of iron and surface treatment retained by using silver nanoparticles as shell. Silver can play an important role as a biocompatible and plasmonic type of material, which can be widely applied in the medical field.

Furthermore, Silver shell nanoparticles can play an important role as its property of resonant oscillation of the outer conduction electrons at the interface which are stimulated by incident light. Using such plasmonic properties, higher SAR of those particles can be obtained by enhancing the particles free motion in biological solution via applying ultrafast femtosecond laser pulses for a short time just before hyperthermia experiment, as representatively illustrated in scheme. 1.

Femtosecond laser exposure to the sample's solution is used to enhance the particles dispersion in solution leading to more efficient localized heating power of the particles in solution. The laser pulses lead to reduction of hydrolyser's volume that surrounds the Fe@Ag core/shell nanoparticles, providing an increase in the particle-particle distance as well as free motion of the particles. The same approach can be applied for, e.g., Fe@Au, or any other Fe/surface plasmonic core/shell structure.

The SAR is measured after laser exposure and the value is increased to 1266 W/g at the same condition of field and frequency of 500 Oe and 164 kHz. This was observed at different field and frequencies with factor of 5-10 increase in SAR values. The data is confirmed by measuring the SAXS (small-angle X-ray scattering) for samples solution before and after laser exposure. SAXS data reveals that the particles after laser exposure shows more dispersion in solution than before laser exposure. This confirms the role of plasmonic surface of the silver shell under applied femtosecond laser to separate the hydrodynamic shell decreasing the inter-particle interaction leading to free movement of isolated particles under applied field and frequency.

The biocompatibility of the selected optimized size of 8.3±1.4 nm is tested for in vitro leukemia tumor cells for different particle's concentrations of 12.5-100 μg/ml. Cytotoxicity data confirm the biocompatibility of the 12.5 and 25 μg/ml doses. Herein, the feasibility of in vitro hyperthermia is performed for the optimized doses under applied therapeutic field and frequency of 400 Oe and 304 kHz followed by cell viability measurements. Data yielded no effect at all for the applied field and frequency on the solvent control and untreated cells. Moreover, there is no effect observed on particle-treated cells in the same day of hyperthermia experiment. However, the particle solutions with cells show apoptosis death for the leukemia cells after 3 and 6 days of in vitro hyperthermia experiment. The findings open new route of independent alternative cancer therapy using magnetic hyperthermia of core-shell Fe@Ag superparamagnetic nanoparticles with plasmonic enhancement.

### EXAMPLES

The following examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### I. Synthesis of Fe@X Core/Shell Nanoparticles

Iron cyanide complex, silver chloride (AgCl), sodium borohydride (NaBH₄) are purchased from Fisher Scientific. All chemical compounds are used as received and without further purification. In all cases, profiling of samples was carried out under ambient conditions.

### Fe@Ag:

Fe@Ag core/shell nanoparticles are synthesized using room temperature chemical wet methods. Briefly, Fe@Ag nanoparticles can be synthesized by coprecipitation of Potassium ferricyanide, silver chloride, sodium borohydride (NaBH4) salts in the presence of ethanol.

Iron Nanoparticles were produced with the aid of strong capping agent at low pH value to prevent particle agglomeration. Pore directing agent was added during the synthesis process. Different types of commonly used pore capping agents were evaluated, including CTAB and PEG. In ambient conditions, PEG was preferred, as it separated easily by sonication and washing.

Synthesis was performed by preparing ethanol solution of the iron cyanide complex, which stirred vigorously for 1 hour to confirm homogeneity and dispersion. Then silver was added to the solution while stirring for 2 hrs. Reduction process proceeded by using sodium borohydride as strong reducing agent and sodium hydroxide as precipitant at ambient conditions after addition of silver. Coating silver over the surface of produced iron nanoparticles prevent the further oxidation. Fe@Ag core shell nanoparticles separated after stirring. The process was optimized via varying the iron salt amount obtaining stable Fe@Ag core/shell nanoparticles with two different sizes of 8 and 13 nm.

### Fe@Au:

To synthesize the Fe@Au core/shell nanoparticles using wet chemical method, potassium ferricyanide is mixed with ethanol, then reduced by sodium borohydride to form iron core. Secondly, gold chloride will be added to the mixture to form and were mixed with ethanol at different Fe:Au ratios. In all cases, profiling of samples was carried out under ambient conditions. Functionalization of Au/Fe Core shell Fe nanoparticles produced with the aid of one of the pore directing agents, tween at low ph. Pore directing agent was added during the synthesis process to control the size and to prevent particle agglomeration. Various types of popular surfactants were compared in this research work including CTAB and PEG.

### II. Nanoparticle Characterization

Fe@Ag structure recognized by [P-Analytical X'PERT MPD] instrument used for X-ray diffraction patterns to capture peaks which characterizes the obtained materials. Morphology studied by scanning electron microscope from Hitachi. Core/shell morphology was determined using transmission electron microscope from JEOL. Vibrating Sample Magnetometer VSM (Quantum Design, 3T Versalab) was applied to study the magnetic characteristics of the particles. Hyperthermia measurements are carried out using D5 hyperthermia system from Quantum design. SAXS measurements were carried out using a Xeuss 2.0 HR SAXS/WAXS system (Xenocs, Sassenage, France) with a Cu source tuned to λ=0.1542 nm and at three sample-to-detector distances of 2500 mm, 1209 mm, and 150 mm which yielded a combined Q-range of 0.003-1.67 Å⁻¹.

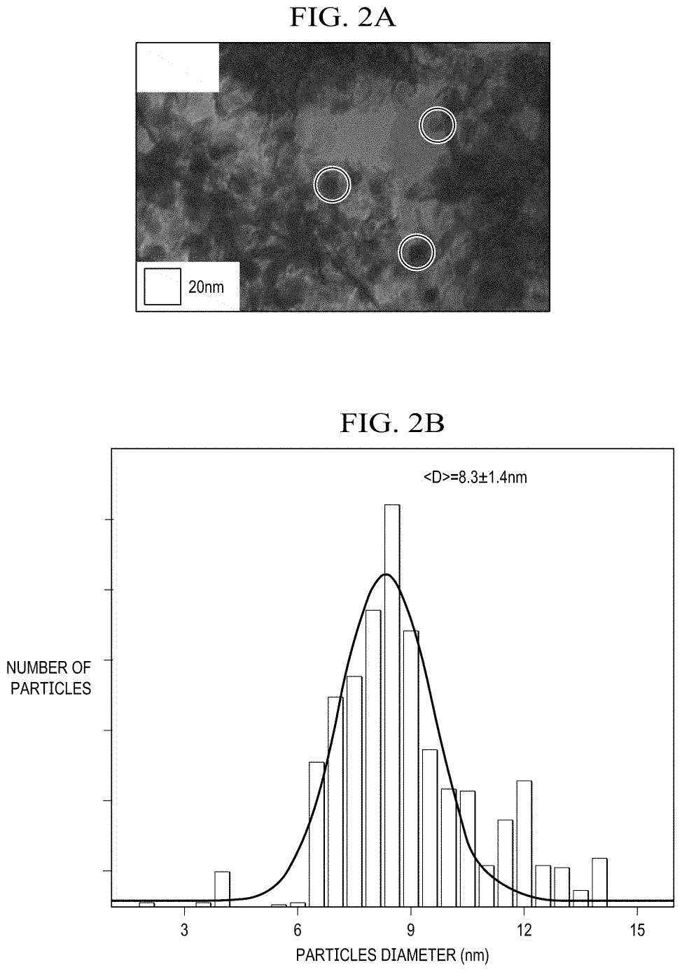

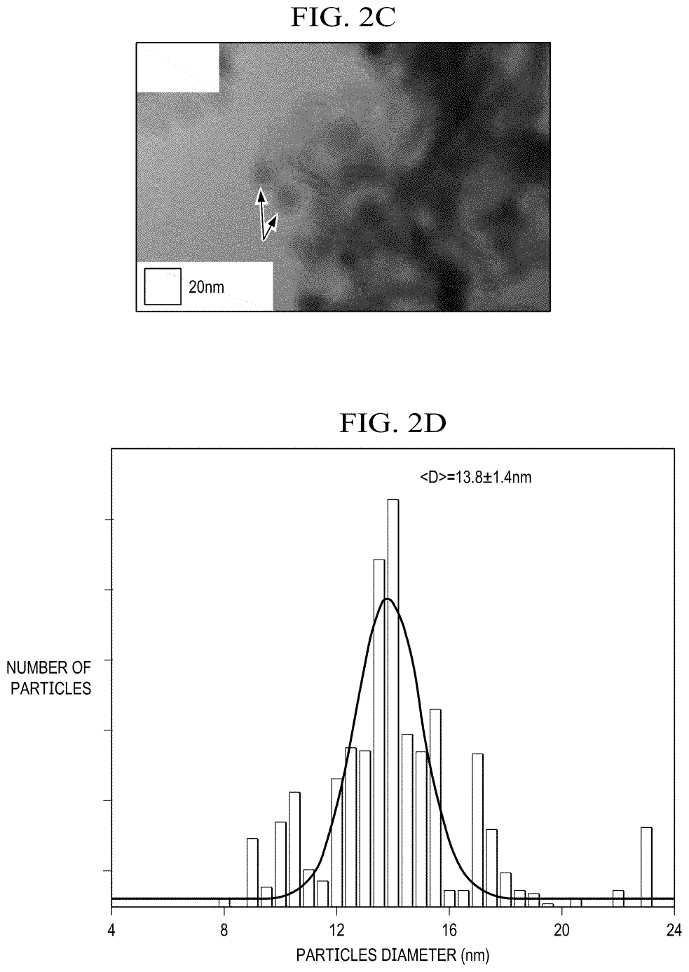

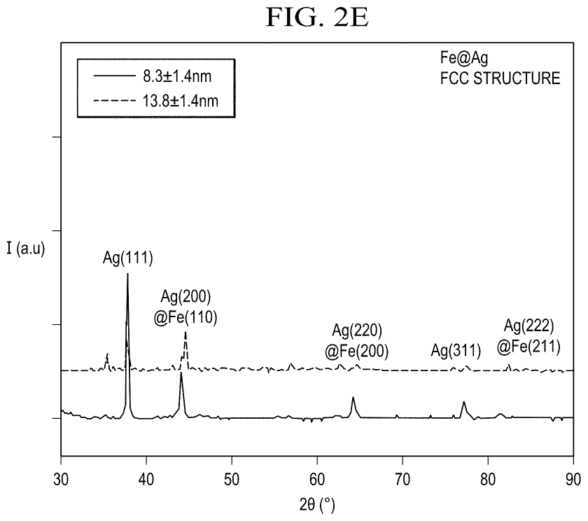

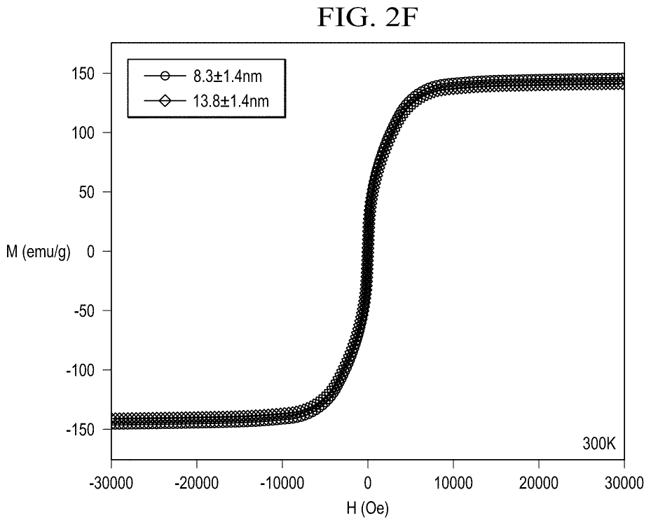

The morphology and size distribution of the synthesized particles were characterized using TEM and SEM respectively (FIG. 2(a-d). The particles show spherical-like shape with narrow size distribution and average size of 8.3±1.4 nm (FIG. 2(a,b) and 13.8±1.4 nm (FIG. 2(c,d).

The TEM images illustrate formation of the core/shell morphology as shown in FIG. 2(a,c). The crystalline structure of the formed core/shell particles were characterized using XRD as shown in FIG. 2(e). The Fe@Ag nanoparticles exhibit face centered cubic (FCC) structure without any related oxides peaks. This confirm the formation of pure iron protected with silver coating shells. The average crystalline size of the iron core was determined from the diffraction peak of (110) plane using Scherrer's Equation to be 4 nm. The magnetic properties of the samples have been investigated by studies of the magnetization dependence on magnetic field up to 3 T at room temperature of 300 K (FIG. 2(e)).

For all samples, the data imply superparamagnetic behavior as indicated with closed hysteresis loop. The superparamagnetic behavior is confirmed by the saturation magnetization (M_{S}) of 145, and 141 emu/g for Fe@Ag nanoparticles with average size of 8.3±1.4 and 13.8±1.4 nm respectively. The superparamagnetic behavior confirms the formation of small particles size below the single domain critical size D_{cr} of Fe that amounts 15 nm. This threshold is the maximum size for which coherent magnetization reversal of a single magnetic domain is feasible. Particles with size smaller than D_{cr}, the coercivity H_{C} decreases rapidly as the particle size decreases.

### III. Heating Effect of the Nanoparticles

In contrast to measurements presented above, the following experiments have been performed by means of dispersed particles in an aqueous solution. In order to prepare the dispersions, distilled water was used as a biocompatible surfactant. The particles were dispersed in water using a sonicator. The concentration of the particles was chosen to be 5 mg/ml for Fe@Ag nanoparticles.

The heating effect of the particles dispersion in alternating (AC) magnetic fields was studied by means of a high frequency generator with water-cooled magnetic coil system. AC magnetic fields with a frequency range of 144-304 kHz and magnetic field strengths of 0-500 Oe were applied to the samples. The temperature of the sample's solution was measured by a fiber-optical temperature sensor. Time-dependent calorimetric measurements at different applied magnetic fields and frequencies for both sizes of 8.3 and 13.8 nm Fe@Ag nanoparticles were measured.

For both samples, a significant heating effect is observed at applied magnetic fields >200 Oe. This heating is usually described in terms of the specific absorption rate (SAR). The SAR expresses the heating ability of a magnetic material and, therefore, the feasibility of a material for application in magnetic hyperthermia. The SAR value is calculated from the initial slope of the T vs.t curves:

$$S A R = C {m}_{act} \vert \frac{d T}{d t} {\vert}_{t = 0}$$

where m_{act} is the mass ratio of the magnetically active material in the solution; and
C the heat capacity of water (C=4.18 Jg⁻¹K⁻¹).

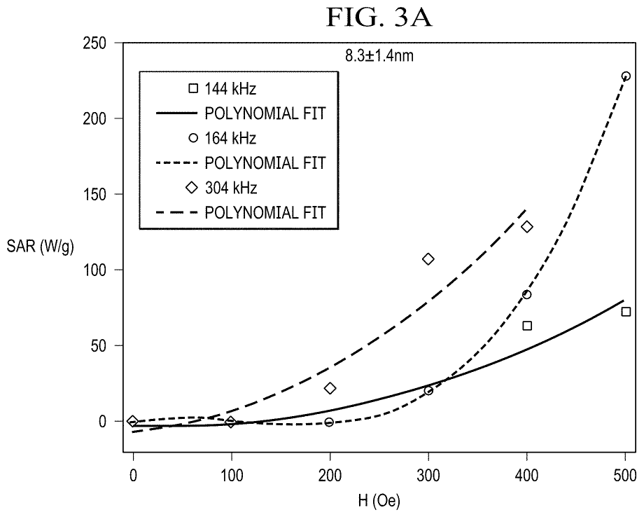

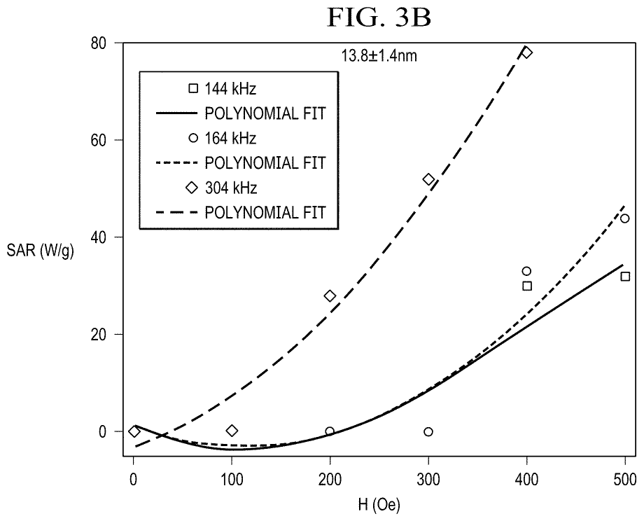

FIG. 3(a, b) shows the magnetic field dependence of the SAR for Fe@Ag for both sizes of 8.3 and 13.8 nm respectively. As seen in FIG. 3 (a, b), SAR increases when the applied magnetic field and frequency increase. The data yield SAR values of 227 and 44 W/g at the maximum applied magnetic field of 500 Oe at 164 kHz, for 8.3 nm and 13.8 nm Fe@Ag nanoparticles, respectively. The observed quadratic field dependence is in agreement with the fact that the dissipated magnetic energy is proportional to H².

Heating of magnetic particles in an alternating magnetic field may be understood in terms of several types of energetic barriers which must be overcome for reversal of the magnetic moments. With decreasing particle size, these barriers decrease and the probability of jumps of the spontaneous magnetization due to the thermal activation processes, as well as SAR, increases. Due to narrow size distribution in the samples, the particles imply only superparamagnetic single domain behavior at room temperature. Hence different heating mechanisms might appear concomitantly from which Neel and Brownian relaxation are expected to be the relevant processes for the observed power absorption.

### IV. Plasmonic Enhancement

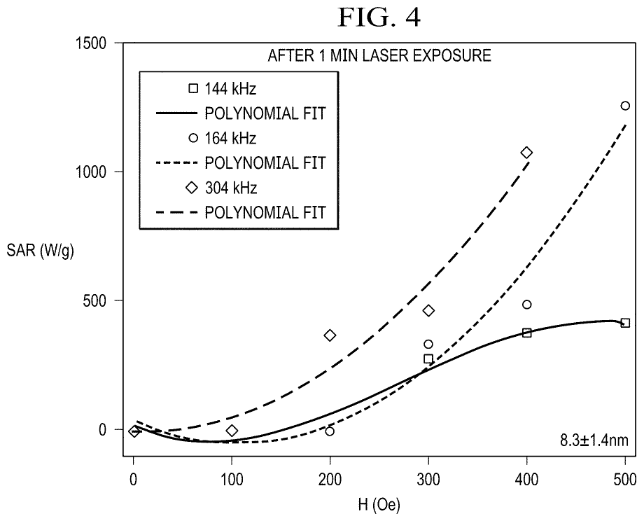

In order to enhance the SAR values of our Fe@Ag nanoparticles using plasmonic surface effect of silver shells, ultrafast femtosecond laser with power of 150 W and wavelength of 710 nm has been applied to the 8.3 nm Fe@Ag sample's solution for short time period. The hyperthermia experiment for the laser-exposed sample has been performed under the same conditions of field and frequencies. The SAR values have been calculated revealing observable increase in magnitude with factor of 5-10 (depending on applied field and frequency) compared to the SAR values without laser exposure (FIG. 4). The data yield SAR of 1266 W/g at maximum magnetic field 500 Oe and frequency of 164 kHz.

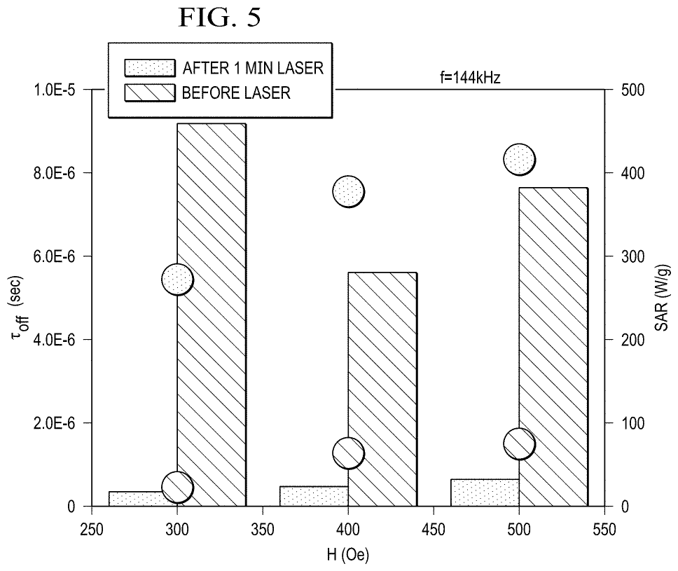

In order to understand the reason behind that observable increase, the effective relaxation time who is responsible for heating mechanism as well as SAR values has been calculated using Neel-Arrhenius equation (FIG. 5). FIG. 5 representatively illustrates effective relaxation time (τ_{eff}) dependence on applied magnetic field magnitude at fixed frequency of 144 kHz. The data illustrates reduction in effective relaxation time after laser exposure with factor of 11 (depending on the applied field and frequency) compared to the measurement before laser exposure.

Since the magnetic moment rotation (Neel relaxation) does not get affected by laser exposure and remains the same, then the only reason for increasing SAR is the particles movement becomes faster in the solution (Brownian relaxation time becomes shorter) after laser exposure. In order to confirm such conclusion, small-angle X-ray scattering (SAXS) measurements were carried out for sample solutions before and after laser exposure.

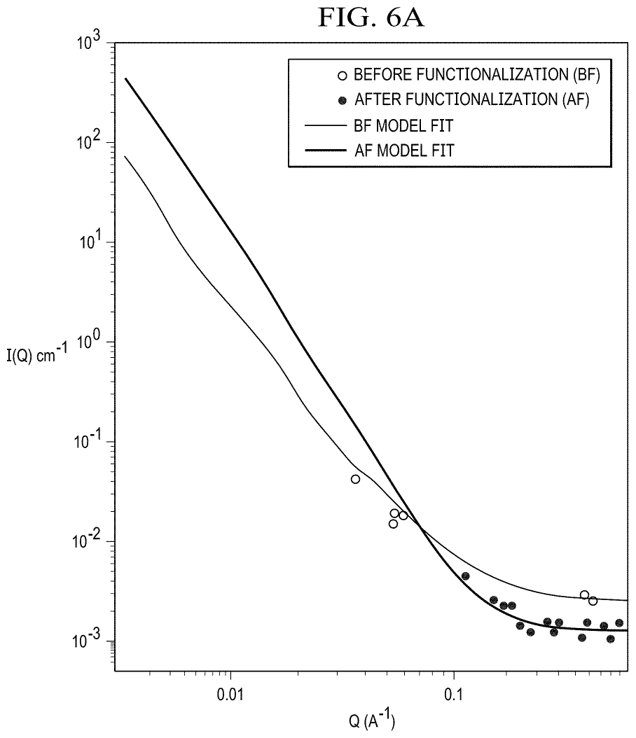

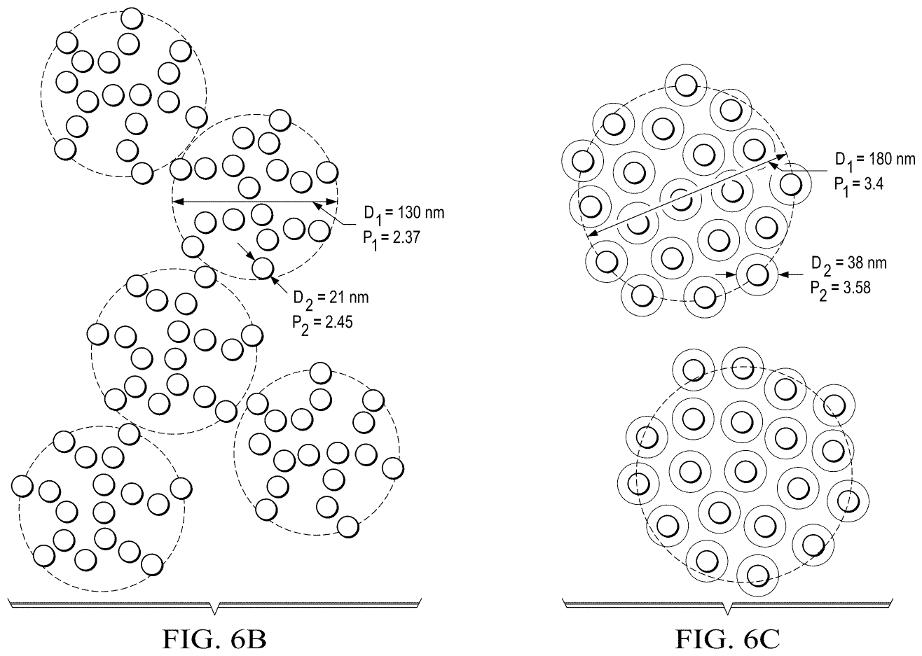

The particle solutions were loaded into nominally 1.0 mm-path length boron-rich thin walled capillaries and sealed with high temperature hot glue. In SAXS, X-rays scattered as function of the scattering angle 2θ, with respect to the transmitted direct beam, are collected on an area detector. During the measurement, laser exposed nanoparticles were observed to have remained dispersed for several hours, whereas non-laser exposed nanoparticles settled to the bottom of the capillary within one hour. Scattering from both samples was observed due to the magnetic nanoparticles, the manner in which they form aggregates, and the larger agglomerates formed by the aggregates. A two-level unified exponential model (henceforth referred to as the UEP model) was applied to the I(Q) SAXS data in order to extract the dimensions of the nanoparticles, aggregates, and fractal dimension of each. FIG. 6A shows a plot of the SAXS experimental data with the UEP model curves overlaid on the data.

Prior to functionalization with laser, the SAXS data is well-described by a nanoparticle with average size of D₂=2*R_{g,2},=21 nm. The nanoparticles are aggregated into mass fractals with dimension d_{m,2}=2.45. The aggregates are clustered into large agglomerates of size D₁=2*R_{g,1},=130 nm, and the agglomerates are also arranged as mass fractals, in this case with dimension d_{m,1}=2.37. The mass fractal character at both length scales indicates that there are significant inter-particle interactions and the individual nanoparticles are not dispersed in the water. The mass fractal value for the aggregate, P₁=2.37, also indicates that agglomerates much larger than the D₁=130 nm size are found in the sample, which correlates well with observation that material does not remain suspended in the solvent for a long time.

Table 1 summarizes size and fractal dimension parameters obtained from the UEP model. SAXS data from the sample which was functionalized by laser

Table 1, sample: After Functionalization shows a larger individual nanoparticle dimension, D₂=38 nm. In this case, the slope P₂=3.58, which indicates that the power law signal is due to scattering from the surface of the nanoparticle, and that there is no longer an interconnected mass fractal structure present. There appears to be an assemblage of loosely coupled particles that measures 90 nm±45 nm and is also not part of a mass fractal network (P₁=3.44 in this case). There is greater uncertainty in the exact dimensions of this assemblage because the signal is near the limits of the instrument's size resolution. The finding that the i=1 and i=2 structural levels in the functionalized system both exhibit surface fractal scattering, rather than mass fractal, indicates that the particles are better dispersed in water and that strong particle-particle interactions are minimized in this sample after laser exposure.

TABLE 1
UEP Model parameters. The i = 2 parameters correspond to the nanoparticle
size and fractal scattering behavior. The i = 1 parameter corresponds
to the aggregate dimensions and their fractal scattering behavior.

| | R_{g1} | D₁ | | R_{g2} | D₂ | |
|---|---|---|---|---|---|---|
| Sample | (nm) | (nm) | P₁ | (nm) | (nm) | P₂ |
| Before | 65 ± 2.2 | 130 ± 4.3 | 2.37 ± 0.09 | 10.3 ± 1.4 | 20.6 ± 2.7 | 2.45 ± 0.06 |
| Function- | | | | | | |
| alization. | | | | | | |
| After | 90.5 ± 45 | 181 ± 90 | 3.44 ± 0.41 | 19.2 ± 1.3 | 38.4 ± 2.6 | 3.58 ± 0.02 |
| Function- | | | | | | |
| alization | | | | | | |

### V. Cytotoxicity

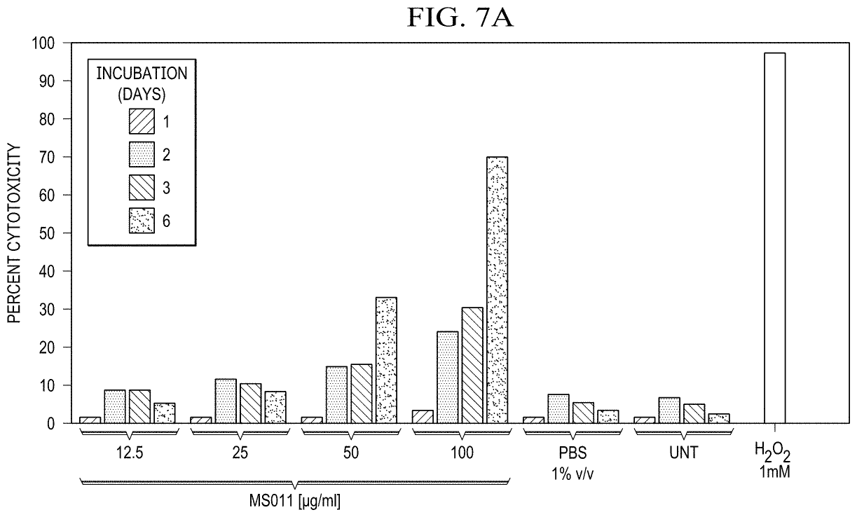

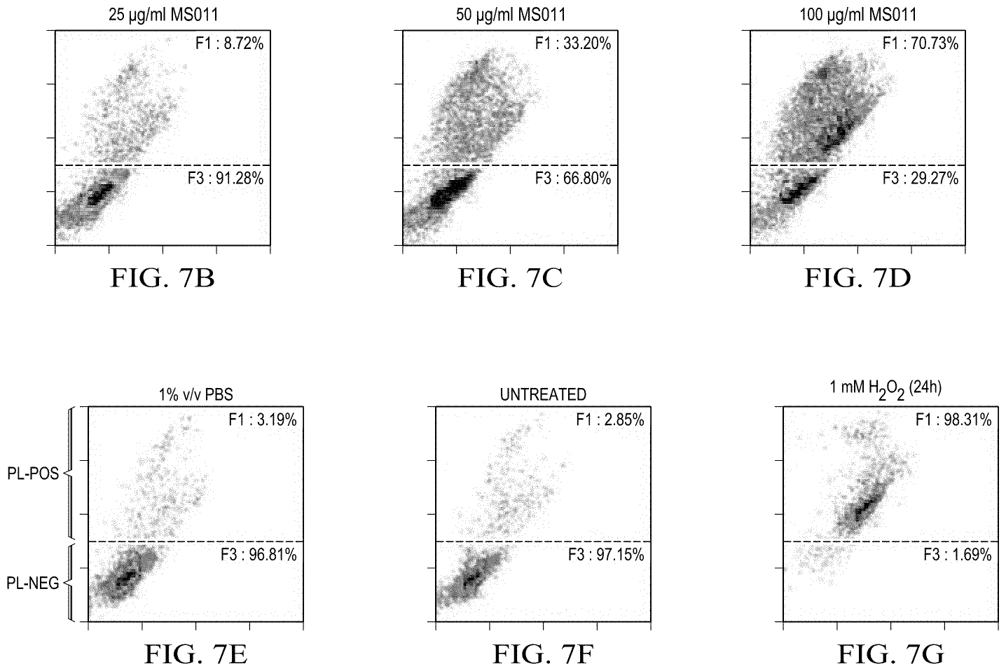

For in vitro studies, Fe@Ag (8 nm size) were tested for their capability to inflict cytotoxicity on human leukemia HL-60 cell line (FIG. 7). Human leukemia HL-60 cell line was acquired from American Tissue Culture Collection (ATCC; CCL-240) and are cells growing in suspension (non-adherent). HL-60 cells were grown by using RPMI 1640 culture media (Corning) supplemented with 20% of fetal bovine serum (Hyclone) and 1× antibiotics; 100 U/ml penicillin and 100 μg/ml streptomycin (Life Technologies). Cultures with a viability of 95% or higher were used. Low cell viability cultures were processed as previously described, to increase the percentage of viable cells (Lema et al. 2011). Consistently, cells were incubated at 37° C. under a humidified 5% carbon dioxide (CO₂) atmosphere using a typical water-jacketed incubator. Cells growing in exponentially phase, around of 60-70% of confluence were collected and seeded on 24-well plate format at a density of 25,000 cells/well in 1 ml of culture media, followed by overnight incubation.

Subsequently, cells were exposed for several days to a concentration gradient of MS011 nanoparticles. Cells were incubated with a concentration gradient (in μg/ml) of 8 nm-Fe@Ag nanoparticles and incubated for 1, 2, 3, and 6 days (FIG. 7(a)).

Cellular cytotoxicity was quantified by using the fluorescent dye exclusion propodeum iodide (PI) and flow cytometry assays. At each indicated incubation time, cells were harvest, stained with 5 μg/ml of fluorescence propidium iodide (PI) reagent and immediately analyzed via flow cytometer (Gallios, Beckman Coulter). Also, unstained and untreated cells, PI-stained untreated cells, as well as H2O2-treated stained cells were used to fine-tune the voltages for the FL1 and FL2 detectors, as well as to adjust the compensation values.

Cells treated with 1% v/v of PBS were used as a solvent control (FIG. 7 (a, e). Untreated cells were included as a negative control for cytotoxicity (FIG. 7 (a, f). Also, cells exposed for 24 h to 1 mM of H₂O₂ were incorporated as a positive control for cytotoxicity (FIG. 7(a, g)).

Two parameters flow cytometer dot plots were obtained using FL1 (x-axis) and FL2 (y-axis) detectors, respectively. Each flow cytometric dot plot was divided into two sections (top and bottom) by a horizontal line: top section corresponds to PI-positive (pos) dead cells, whereas bottom section corresponds to PI-negative (neg) living cells (Varela-Ramirez et al. 2011; Santiago-Vazquez et al. 2016; Ruiz-Medina et al. 2019). Around 10,000 events (cells) were acquired per sample and analyzed via Kaluza software (Beckman Coulter).

Representative two parameters flow cytometer dot plots are depicted in FIG. 7 panels b to g, using FL1 (x-axis) and FL2 (y-axis) detectors, respectively. Dot plots of cells treated for 6 days with a concentration gradient of Fe@Ag: B, 25 μg/ml; C, 50 μg/ml; and D, 100 μg/ml. The PI-positive (pos) HL-60 cells (dead) are included in the top and the PI-negative (neg) unstained cells (living) in the bottom section of each dot plots, respectively.

The data show minimum toxicity for particles concentrations of 12.5 and 25 μg/ml, while for particles with concentration of 50 and 100 μg/ml, the data imply higher cytotoxicity for the cells. Therefore, a representative dose of 8 nm Fe@Ag nanoparticles to be utilized for hyperthermia is 12.5 and 25 μg/ml.

### VI. In Vitro Hyperthermia

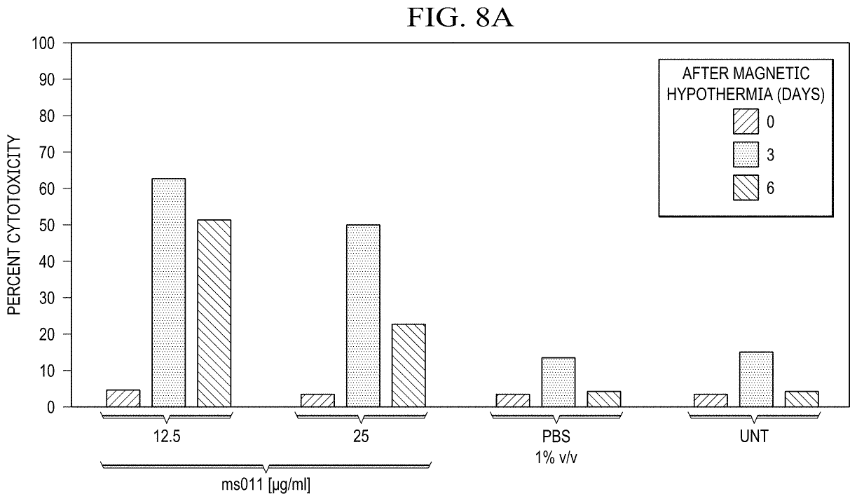

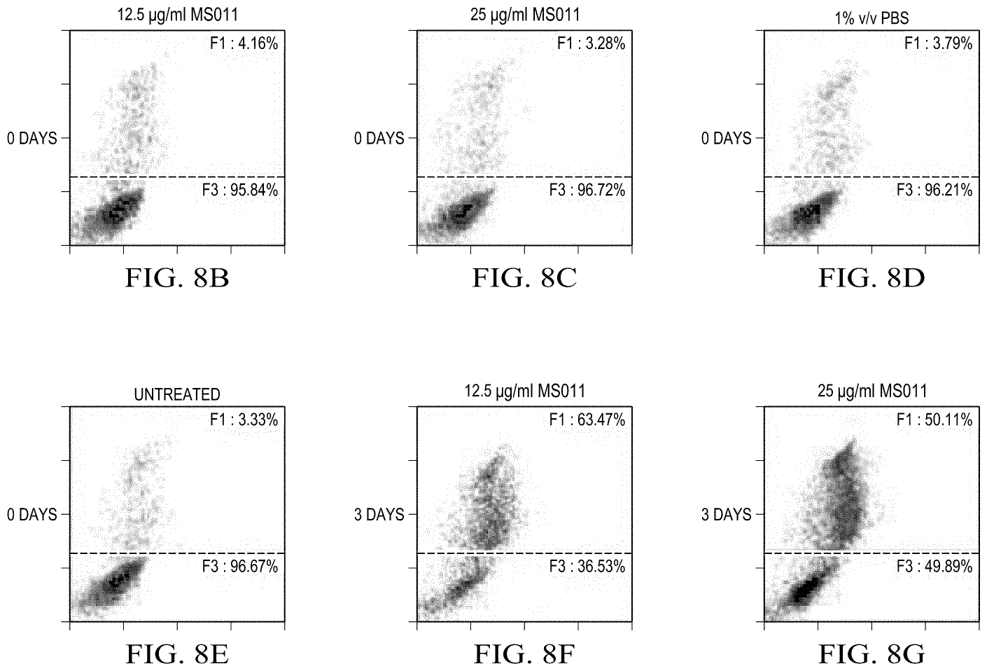

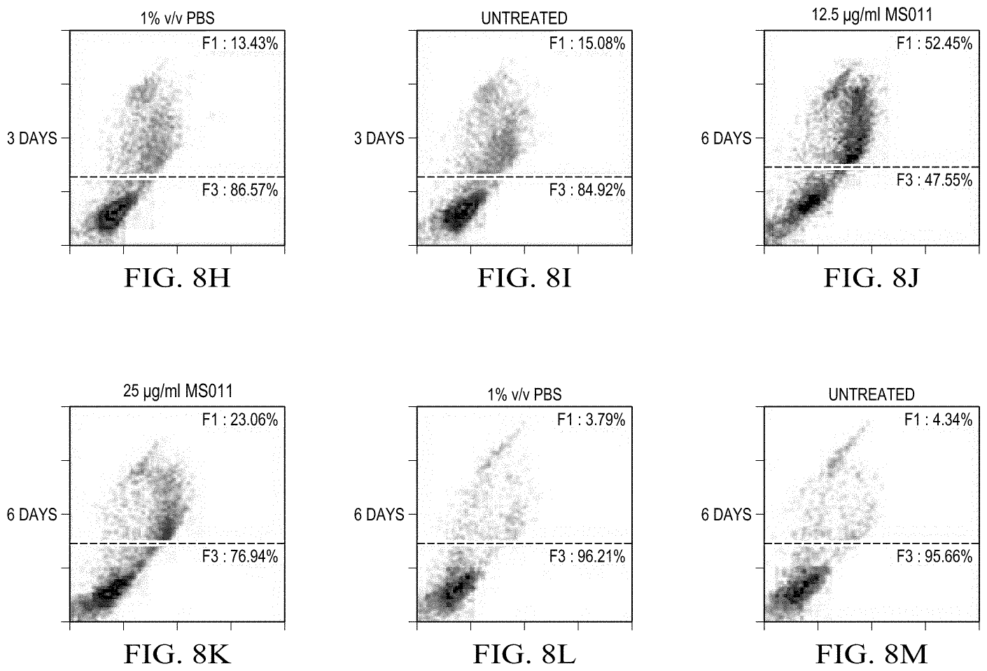

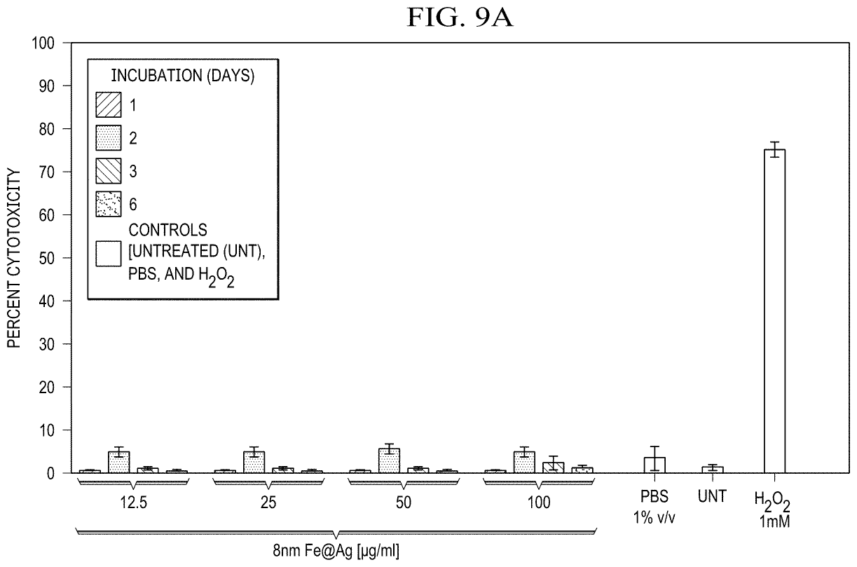

In order to take a step for treatment, the feasibility of 8 nm Fe@Ag for in vitro hyperthermia has been tested within the therapeutic limit of applied field and frequency. The particles with concentrations 12.5 and 25 μg/ml plus PBS as solvent control and untreated cells as negative control were prepared for the experiment. The hyperthermia conditions for all the samples is fixed to be 400 Ce, 304 kHz, and 30 min for applied field, frequency and exposure time, respectively. The cell viability measurement was performed after the hyperthermia experiment immediately (0 days), 3, and 6 days as shown in FIG. 8. The data imply no cytotoxicity for the cells in the 0 days measurement after hyperthermia (FIG. 8 (a-d). For the cell viability measurements after 3 days, and 6 days, the particles show an apoptosis death to the cells as shown in FIG. 8 (f-m).

### VII. Cytotoxicity

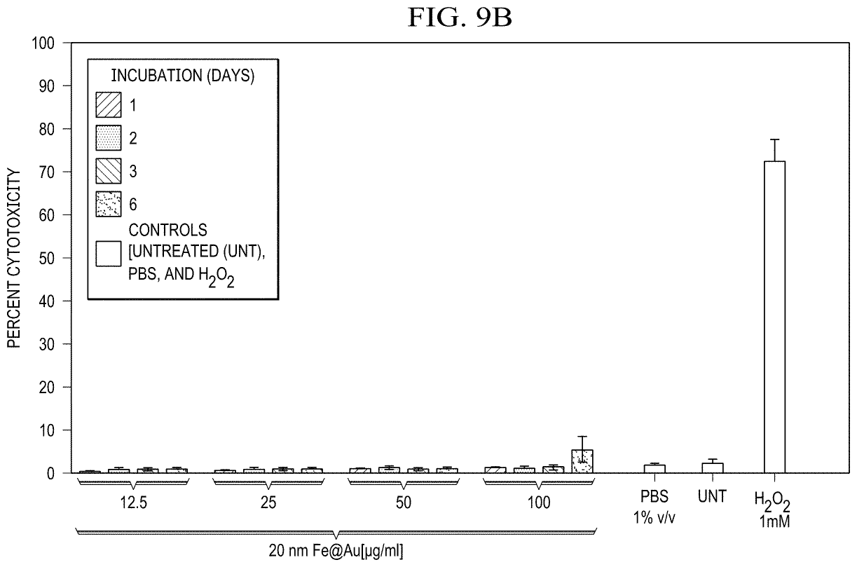

The potential cytotoxic activity of 8 nm Fe@Ag and (b) 20 nm Fe@Au magnetic nanoparticles were tested for their capability to inflict cytotoxicity on MDA-MB231 breast cancer cell line containing different concentrations of Fe@Ag or Fe@Au (FIG. 9(a-b)), incubated for 1, 2, 3, and 6 days.

The 8 nm Fe@Ag and 20 nm Fe@Au MNP did not exhibit any significant cytotoxicity at any concentration and incubation time tested. Moreover, after testing all the MNP at 12.5 μg/ml and 25 μg/ml concentrations, for 1 to 6 days of incubation periods. The cytotoxic values were similar to those observed for untreated, and solvents control cells revealing no cytotoxicity was detected.

These findings open new route for new alternative cancer therapy using plasmonic enhanced magnetic hyperthermia of Fe@Ag nanoparticles.

The descriptions of the various embodiments of the present invention have been presented for purposes of illustration, but are not intended to be exhaustive or limited to the embodiments disclosed. Not all embodiments will include all of the features described in the illustrative examples. Further, different illustrative embodiments may provide different features as compared to other illustrative embodiments. Many modifications and variations will be apparent to those of ordinary skill in the art without departing from the scope and spirit of the described embodiment. The terminology used herein was chosen to best explain the principles of the embodiment, the practical application or technical improvement over technologies found in the marketplace, or to enable others of ordinary skill in the art to understand the embodiments disclosed here.

What is claimed is:

1. A composition comprising:
M@X core/shell magnetic nanoparticles formed by coprecipitation of an M-salt, an X-salt, and sodium borohydride salt in ethanol, and applying laser energy to form the agglomerates having an average particle diameter greater than 100 nanometers, wherein:
M comprises Fe, Co, Ni, or combinations thereof; and
X comprises Ag, Au, or combinations thereof.

2. The composition of claim 1, wherein the M@X core/shell nanoparticles have an average size of about:
8.3 nm; or
13.8 nm.

3. The method of claim 1, wherein the M@X core/shell nanoparticles have a face-centered cubic (FCC) structure.

4. The method of claim 1, wherein the average crystalline size of the iron core is about 4 nm.

5. A method comprising:
forming an M@X core/shell nanoparticle by coprecipitation of an M-salt, an X-salt, and sodium borohydride salt in ethanol, wherein:
M comprises Fe, Co, Ni, or combinations thereof; and X comprises Ag, Au, or combinations thereof; and
applying laser energy to form the agglomerates having an average particle diameter greater than 100 nanometers.

6. The method of claim 5, further comprising:
stirring an ethanol solution of iron cyanide complex to achieve substantial homogeneity and substantial dispersion of the iron cyanide complex.

7. The method of claim 6, further comprising:
adding silver or a silver compound to the ethanol solution to form Fe@Ag core/shell nanoparticles; or
adding gold or a gold compound to the ethanol solution to form Fe@Au core/shell nanoparticles.

8. A method of using nanoparticles to generate localized heat, the method comprising:
applying laser energy to a solution of M@X core/shell nanoparticles to form the agglomerates having an average particle diameter greater than 100 nanometers, wherein X is one of Ag or Au; and
after applying laser energy, applying oscillating magnetic energy to the solution of M@X core/shell nanoparticles.

9. The method of claim 8, wherein the laser energy comprises a femtosecond laser.

10. The method of claim 9, wherein the femtosecond laser has a power of about 150 W.

11. The method of claim 9, wherein the femtosecond laser has a wavelength of about 710 nm.

12. The method of 8, wherein the oscillating magnetic energy has a magnetic field strength of about 500 Oersted.

13. The method of claim 12, wherein the oscillating magnetic energy has a frequency of about 164 kHz.

14. The method of claim 8, wherein the localized heat comprises a heating power from about 227 W/g to about 1266 W/g.

15. A method for treating abnormal cell growth in a mammal, the method comprising:
applying laser energy to the solution of M@X core/shell nanoparticles so administered to form the agglomerates having an average particle diameter greater than 100 nanometers, where X is one of Ag or Au;
administering to said mammal, in the vicinity of the abnormal cell growth, a solution of M@X core/shell nanoparticles;
after applying laser energy, applying oscillating magnetic energy to the solution of M@X core/shell nanoparticles in the vicinity of administration.

16. The method of claim 15, wherein the laser energy comprises a femtosecond laser.

17. The method of claim 16, wherein the femtosecond laser has a power of about 150 W.

18. The method of claim 17, wherein the femtosecond laser has a wavelength of about 710 nm.

19. The method of 15, wherein the oscillating magnetic energy has a magnetic field strength of about 500 Oersted.

20. The method of claim 19, wherein the oscillating magnetic energy has a frequency of about 164 kHz.

21. The method of claim 15, wherein localized heat produced in the vicinity of administration of the M@X core/shell nanoparticles comprises from about 227 W/g to about 1266 W/g of heat.
